(19) United States
(12) Patent Application Publication
(10) Pub. No.: US 2024/0091114 A1
(43) Pub. Date: Mar. 21, 2024

(54) Make-up Composition With Gloss and Lasting Properties

(71) Applicant: L V M H RECHERCHE, Saint-Jean De Braye (FR)

(72) Inventors: Elodie Champredonde, Saint-Jean De Braye (FR); Valérie Bouchard De La Poterie, Saint-Jean De Braye (FR)

(21) Appl. No.: 18/268,650

(22) PCT Filed: Dec. 23, 2021
(86) PCT No.: PCT/FR2021/052442

§ 371 Date: Jun. 21, 2023

(30) Foreign Application Priority Data
Dec. 23, 2020 (FR) FR2014068

(51) Int. Cl. A61K 8/31; A61K 8/58; A61K 8/892; A61K 8/898; A61Q 1/06

(52) U.S. Cl. CPC A61K 8/31; A61K 8/585; A61K 8/892; A61K 8/898; A61Q 1/06; A61K 2800/31; A61K 2800/43

(57) ABSTRACT

The present invention relates to a homogeneous cosmetic composition for make-up of keratin materials, in particular the lips, comprising, in a physiologically acceptable medium, at least: —one or more, linear or branched, C8-C19 alkanes; —one or more phenyl silicone oils, the refractive index of which at 25° C. is greater than 1.45, preferably greater than 1.47; —a silicone-polyurethane polymer; —and optionally, in addition, at least one pigment.

### FIELD OF THE INVENTION

The present invention concerns the cosmetic field and, in particular, cosmetic compositions for applying make-up to keratin materials, in particular the lips.

### STATE OF THE ART

Current lip make-up products are often categorized as products exhibiting good colour-lasting or as glossy products. For a dual effect of colour lasting and gloss, the products are generally in the form of a combination of formulas requiring a two-step operation to obtain a film with the desired properties, with a first base formula, also called ‘base coat’, applied on the lips to provide colour and lasting, and a second formula to apply over the first, also called ‘top coat’, generally colourless and providing a glossy film.

But there remains the need to develop products that apply in a single step, having both colour lasting performance and gloss performance and that are homogeneous (homogenous colour). In addition to the simplicity of use, the application in a single step also makes it possible to have a thinner film (a single layer on the lips so less material is deposited).

The Applicant has precisely developed a homogeneous, single-step composition which makes it possible to meet this expectation, by combining a silicone-polyurethane polymer, one or more linear or branched C8-C19 alkanes and one or more phenyl silicone oils.

This combination makes it possible to have an immediate gloss greater than or equal to the products on the market, an improved gloss lasting over time, a product that is applied by a single operation and is macroscopically homogeneous.

This composition is advantageous as a make-up composition for keratin materials, in particular the lips.

### DISCLOSURE OF THE INVENTION

A first aspect of the invention is therefore a homogeneous cosmetic composition for caring for and/or applying make-up to keratin materials, in particular the lips, comprising, in a physiologically acceptable medium, at least:
one or more linear or branched C8-C19 alkane(s);
one or more phenyl silicone oil(s) whose refractive index at 25° C. is greater than 1.45, preferably greater than or equal to 1.47;
a silicone-polyurethane polymer;
and, optionally, also at least one pigment.

‘Keratin materials’ are understood especially to mean the skin and/or lips, hair, eyelashes and nails. According to a particular embodiment, the composition of the invention is applied to the lips.

The invention also relates to a cosmetic process for caring for and/or applying make-up to keratin materials, in particular the lips, comprising the application onto said keratin materials, in particular the lips, of a composition according to the invention.

### DETAILED DESCRIPTION OF THE INVENTION

Thus, a first aspect of the invention is therefore a homogeneous cosmetic composition for caring for and/or applying make-up to keratin materials, in particular lips, comprising, in a physiologically acceptable medium, at least:
one or more linear or branched C8-C19 alkane(s);
one or more phenyl silicone oil(s) whose refractive index at 25° C. is greater than 1.45, preferably greater than or equal to 1.47;
a silicone-polyurethane polymer;
and, optionally, also at least one pigment.

‘Homogeneous’ is understood to mean that the composition of the invention has a visually homogeneous appearance, i.e., that each of the phases cannot be individualized with the naked eye in the composition which thus seems to consist of only a single phase.

According to a particular embodiment, the oil phase in which the silicone- polyurethane polymer is solubilized comprises total contents of alkane oil(s) and, respectively, of phenyl silicone oil(s), such that the oily solution after mixing and incorporation of the silicone-polyurethane polymer is translucent or transparent.

Reference will be made to ‘mixture of alkanes/phenyl silicone oils/silicone-polyurethane polymer capable of forming a translucent or transparent oily solution’ or ‘transparent or translucent ternary mixture’.

‘Translucent or transparent oily solution’ or ‘translucent or transparent ternary mixture’ is understood to mean that the solution or mixture is not cloudy or opaque, and allows light to pass through and clearly show objects behind it.

According to a particular embodiment, the mixture comprises isododecane (branched alkane), a trimethyl pentaphenyl trisiloxane (and, optionally, a diphenylsiloxy phenyl trimethicone) and a silicone-polyurethane polymer in proportions defined for obtaining a translucent or transparent oily solution, leading to the production of a homogeneous composition after addition of the other ingredients of the composition.

The person skilled in the art in the cosmetic field will thus define the optimum contents of each of the compounds of the ternary mixture according to their knowledge of the characteristics specific to each compound (in particular solubility, molecular weight) and its behaviour as a binary mixture of alkane/phenyl silicone oil and then ternary alkane/phenyl silicone oil/polymer, to obtain a translucent or transparent oily solution.

According to a particular and preferred embodiment, the composition of the invention is an anhydrous composition.

‘Anhydrous’ is understood to mean in particular that water is preferably not added to the compositions but may be present in trace amounts in the various compounds used in the compositions. In particular, the composition according to the invention comprises less than 4% by weight of water, preferably less than 3%, preferably less than 2%, more preferably less than 1%, even more preferably less than 0.5% by weight of water, relative to the total weight of the said composition, or is completely free of water.

According to a particular and preferred embodiment, the composition of the invention is a liquid and anhydrous composition.

The composition of the invention is a glossy composition.

In particular, the gloss value of a deposit of a cosmetic composition is measured by using a gloss meter and expressed in gloss units (GU). Reference will be made equally to the gloss value of the composition or the gloss value of the deposit on the skin or the lips. The gloss value of a composition may, in particular, be measured according to the following protocol.

In order to measure the gloss of a product to be tested, a light beam of constant intensity is directed at a predetermined angle onto the surface to be tested, and the quantity of light reflected at the same angle is then measured. This specular reflection is measured using a glossmeter. Depending on the gloss level, different angles of reflection are used. The gloss measurement is based on the amount of light reflected from a surface relative to a polished glass reference standard and is expressed in gloss units (GU).

The measurement is normalized relative to an internal standard and reduced to a value out of 100: For this standard, the measurement value is set at 100 gloss units (calibration). The closer the measured value is to 100, the glossier the sample.

The amount of light reflected on the surface depends on the angle of incidence and the properties of the surface itself.

The gloss level is divided into three categories: matte (gloss value less than 10 GU), semi-gloss (gloss value between 10 and 70 GU) or high gloss (gloss value greater than 70 GU). To determine the appropriate measurement angle, a glossmeter with an angle of incidence of 60° is used.

In the context of the present invention, the gloss value of the composition is generally greater than 70 GU, in particular greater than or equal to 75 GU, or even greater than or equal to 80 GU.

In addition, the composition of the invention is advantageously a single-step composition, to provide colour, shine and lasting.

The composition of the invention also comprises a silicone-polyurethane polymer as defined below. According to a particular embodiment, the composition of the invention comprises a silicone-polyurethane polymer in an active material content (dry matter) ranging from 14 to 25%, preferably from 16 to 20%, by weight relative to the total weight of the composition.

Silicone-Polyurethane Polymer ‘Silicone-polyurethane polymer’ according to the invention is understood to mean any polymer comprising organosiloxane units and urethane bonds.

According to a particular embodiment, the silicone-polyurethane polymer according to the invention is the reaction product of a polyorganosiloxane functionalized by hydroxyl groups, preferably comprising at least two hydroxyl groups, with a diisocyanate compound.

Such a polyorganosiloxane functionalized by hydroxyl groups corresponds to formula (1)

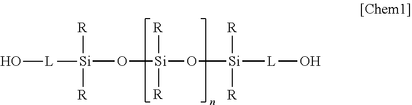

wherein R is independently chosen at each occurrence from a hydrogen atom, a hydroxyl group, and optionally substituted hydrocarbon groups containing from 1 to 10 carbon atoms, and, in particular, from substituted or unsubstituted alkyl, alkenyl, alkynyl, aryl, aryl-alkyl or alkyl-aryl; preferably, R is chosen from optionally substituted linear, cyclic or branched C1-C6 alkyl or alkenyl groups, including, without limitation, methyl, ethyl, propyl, isopropyl, butyl, isobutyl, t-butyl, amyl, hexyl, cyclohexyl, vinyl, C1-C8 allyl or aryl, aryl-alkyl or alkyl-aryl, including, without limitation, phenyl, benzyl, tolyl, xylyl;
wherein each of the above R groups may be optionally substituted by one or more heteroatoms, including oxygen, nitrogen, phosphorus and a halogen, in particular fluorine, as illustrated by fluoroalkyl (perfluoroalkyl) groups such as mono-, di- and tri-fluoromethyl, perfluorophenyl, and C1-C6-substituted alkyl amino groups, including those of the formula —(CH₂)₁₋₆—NR^{N}₂ and —(CH₂)₁₋₆—NR^{N}— (CH₂)₁₋₆-NR^{N}₂ wherein RN is typically hydrogen, but may also be a methyl, ethyl, propyl group, and the like; polyether groups, including, without limitation, polyethylene oxide groups having the formula —(CH₂CH₂O)ₙ—, propylene oxide groups having the formula —(CH(CH₃)CH₂O)ₙ₋ and combinations thereof; and amine oxide, 15 phosphate, hydroxyl, ester and/or carboxylate functions, or the like; or
wherein R may comprise an additional—L-OH group;
   wherein L represents either a bond or a linking group; preferably L is a linking group selected from divalent hydrocarbons having from 1 to 10 carbon atoms, including a divalent alkyl, alkenyl, alkynyl, aryl, alkyl-aryl or aryl-alkyl group, such as, for example, C1-C10 alkyl, including, without limitation, bivalent groups of the formula —(CH₂)₁₋₁₀-, preferably —(CH₂)₁₋₆-, and more preferably, L is —CH₂CH₂CH₂-;
and where n is a whole number between 0 and 5000, preferably between 1 and 200, more preferentially between 10 and 100, and more preferentially still between 10 and 50.

Preferably, R represents at least one or more occurrences of a methyl group, more preferentially, R represents a methyl group at all or substantially all occurrences, which means that R represents a methyl group at more than 90% of occurrences, especially more than 95% of occurrences, or even more than 98% of occurrences. According to one embodiment of the invention, the polyorganosiloxane functionalized by hydroxyl groups comprises a polymethylsiloxane corresponding, for example, to the structure of formula Ia:

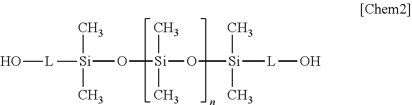

wherein L and n are as defined previously.

According to a preferred embodiment of the invention, the polyorganosiloxane functionalized by hydroxyl groups comprises a polymethylsiloxane corresponding, for example, to the structure of formula Ib:

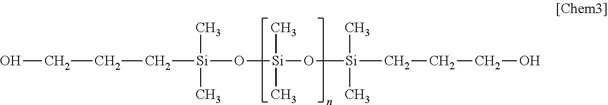

where n is as defined above.

The diisocyanate may especially correspond to the formula O═C═N—R¹—N═C═O, where R¹ is a divalent hydrocarbon group containing from 1 to 20 carbon atoms, optionally including 20

substituted with one or more heteroatoms, and in particular R¹ may be selected from optionally substituted cyclic alkyl, alkenyl, alkynyl, aryl, alkyl-aryl or aryl alkyl, linear, cyclic or branched, including, without limitation:
i. a group of formula:

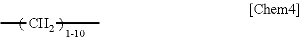

ii. a group of formula:

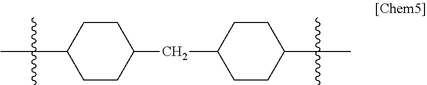 [SMILES: *C1CCC(CC2CCC(*)CC2)CC1]

iii. a group of formula:

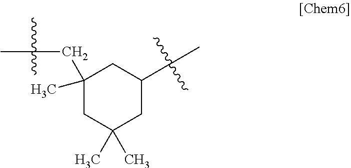 [SMILES: *CC1(C)CC(*)CC(C)(C)C1]

iv. a group of formula:

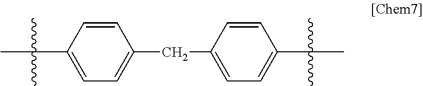 [SMILES: *c1ccc(Cc2ccc(*)cc2)cc1]

v. a group of formula:

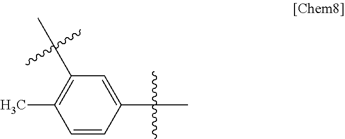 [SMILES: *c1ccc(C)c(*)c1]

and combinations thereof.

The diisocyanates which may be suitable in the context of the invention comprise, but are not limited to, toluene diisocyanate; methylene diphenyl diisocyanate, comprising 2,2′-MDI, 2,4′-MDI and 4,4′-MDI; 1,6-hexamethylene diisocyanate; isophorone diisocyanate; dicyclohexylmethane 4,4′-diisocyanate; xylene diisocyanate; cyclohexane diisocyanate; 3,3′-dimethyl-4,4′-diphenylmethane diisocyanate; p-phenylene diisocyanate; m-phenylene diisocyanate; 4,4′-isopropylidene dicyclohexyl isocyanate; and equivalents thereof.

In a preferred embodiment, the diisocyanate is chosen from the group consisting of 1,6-hexamethylene diisocyanate, dicyclohexylmethane 4,4′-diisocyanate, isophorone diisocyanate, and combinations thereof.

In one embodiment, the diisocyanate comprises or essentially consists of 1,6-hexamethylene diisocyanate.

In another embodiment, the diisocyanate comprises or essentially consists of isophorone diisocyanate.

In still another embodiment, the diisocyanate comprises or essentially consists of dicyclohexylmethane-4,4′-diisocyanate, this embodiment being particularly preferred.

Thus, according to a particular embodiment, the silicone-polyurethane polymer is the reaction product of a polyorganopolysiloxane functionalized by hydroxyl groups, in particular corresponding to the structure of formula (I)

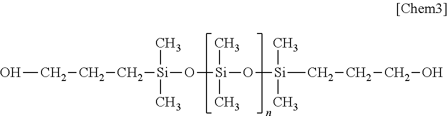

wherein n is a whole number between 0 and 5000, preferably between 1 and 200, more preferentially between 10 and 100, and more preferentially still between 10 and 50;
with a diisocyanate compound, in particular chosen from the group consisting of 1,6-hexamethylene diisocyanate, dicyclohexylmethane 4,4′-diisocyanate, isophorone diisocyanate, and combinations thereof.

According to a preferred embodiment, the silicone-polyurethane polymer according to the invention comprises recurring units derived from polyorganosiloxane functionalized by hydroxyl groups and from diisocyanate in the form of an alternating AB copolymer, where the A unit has the structure of formula II:

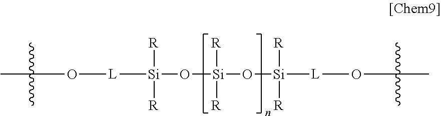

wherein R, L and n are as defined above in relation to formula I, Ia, Ib and Ie, and wherein the B unit has the structure of formula III:

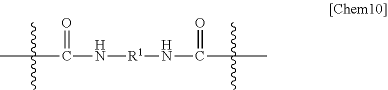

where R1 is as defined above, and wherein the A and B units are arranged in a linear, branched or cyclic, and preferably linear, configuration.

The polymer may also comprise branching or grafting points in the polyorganosiloxane in which one or more R groups in formula I or II is a group IV such as:

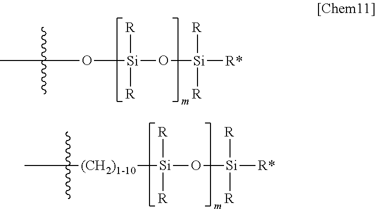

wherein R is as defined for formula I, and R* may represent an -L-O group further coupling the side chain of a B unit of formula III, which in turn may be further coupled to the A unit of formula II, and so on; alternatively, R* may represent L-OH, an R group as defined previously, or a terminal group.

When the polyorganosiloxane contains branching or grafting points of this type, they may be present in the form of T- or Q-type grafts, where T indicates that only one R group on the Si atom is a polyorganosiloxane chain, as indicated above, and Q indicates that two R groups are polyorganosiloxanes.

These types of polyorganosiloxane compounds are referred to as a copolymer of silicone-polyurethane polymer and of a branched or grafted T resin or Q resin.

The silicone-polyurethane polymers may also be prepared from functionalized isocyanate prepolymers. For example, an isocyanate prepolymer may be a difunctional or multi-functional polyorganosiloxane isocyanate, such as the polyorganosiloxane diisocyanate represented below in formula V:

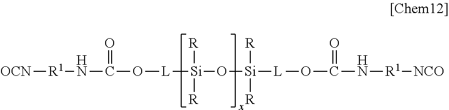

wherein R, R¹ and L are as defined above, and where x is an integer between 0 and 5000, preferably between 1 and 200, more preferentially between 10 and 100, and, more preferably still, between 10 and 50.

The prepolymer may also be multifunctional by introducing additional isocyanate groups bearing one or more R groups. The functionalized isocyanate prepolymer is reacted with a hydroxyl functionalized polyorganosiloxane to obtain a compound of formula I or a multifunctional analogue thereof.

The prepolymer according to formula V generally has a molecular weight ranging from 4000 to approximately 15,000 Daltons. The prepolymer according to formulas I, Ia and Ib, for its part, typically has a molecular weight ranging from 250 to approximately 15,000 Daltons.

In one embodiment of the invention, the silicone-polyurethane polymer is free or substantially free of polyalkylene glycol subunits, especially polyethylene glycol (PEG) or polypropylene glycol (PPG).

‘Substantially free of’is understood to mean that the polymer comprises less than approximately 1% by weight, preferably less than approximately 0.5% by weight and more preferably less than approximately 0.1% by 20% by weight of polyalkylene glycol subunits.

In a preferred embodiment, the silicone-polyurethane polymer used in the cosmetic compositions of the invention is a linear polymer comprising the reaction product of formula Ib with a diisocyanate chosen from the group consisting of 1,6-hexamethylene diisocyanate, dicyclohexylmethane 4,4′-diisocyanate, isophorone diisocyanate, and combinations thereof.

Such a silicone-polyurethane polymer is, for example, available from Siltech Corporation in the form of a premix in isododecane, under the commercial reference SILMER UR 5050 (45% by dry matter of polymer in isododecane).

According to another embodiment, a silicone-polyurethane polymer is used in the form of a premix in a hemi-squalane.

According to another embodiment, a silicone-polyurethane polymer is used in the form of a premix in a linear C9-C16 alkane or a mixture of linear C9-C16 alkanes, preferably a mixture of n-nonane and n-dodecane (C9-C12, VEGELIGHT SILK) or a mixture of n-undecane and n-tridecane (C11-C13, CETIOL ULTIMATE).

According to a particular embodiment, the silicone-polyurethane polymer according to the invention will be present in the composition of the invention in a content ranging from 14 to 25% by weight of active material (dry matter) of polymer, preferably, from 16 to 20% by weight of polymer active material relative to the total weight of the composition.

### Oil Phase

The oil phase of the composition of the invention comprises at least one or more linear or branched C8-C19 alkanes and one or more phenyl silicone oils having a refractive index of at least 1.45, preferably at least 1.47, in particular, whose refractive index at 25° C. is greater than 1.45, preferably greater than or equal to 1.47.

As illustrated in the examples, the Applicant has demonstrated the role of volatile hydrocarbon oils, in particular, linear or branched C8-C19 alkanes in the formulation of the composition of the invention and, in particular, the solubilization of the polyurethane silicone polymer and the incorporation of the phenyl silicone oil to obtain a homogeneous composition.

These linear or branched C8-C19 alkanes, used in a content defined relative to the active material content of the silicone-polyurethane polymer, make it possible to disperse said silicone-polyurethane polymer well and thus facilitate mixing it with the other ingredients of the composition and, in particular, the phenyl silicone oil with a refractive index greater than 1.45.

### Volatile Hydrocarbon Oil (Linear and Branched C8-C19 Alkanes)

‘Oil’ is understood to mean a fatty substance which is liquid at 25° C. and atmospheric pressure.

‘Hydrocarbon-based oil’ is understood to mean an oil mainly comprising carbon and hydrogen atoms and, optionally, one or more functions chosen from hydroxyl, ester, ether and carboxylic functions. These oils are, in particular, free of —Si—O— groups.

‘Volatile oil’ according to the invention is understood to mean an oil which has lost more than 20% by mass of its mass at 15 minutes, more than 40% by mass of its mass at 30 minutes and more than 70% by mass of its mass at 60 minutes, according to the following protocol:

20 mg of the oil to be studied is weighed on a 5 cm×5 cm PMMA plate using a micropipette and a precision balance. This material is spread by finger over the entire plate. The plate is then deposited in a ventilated enclosure thermostatically controlled at 25° C. and 50% humidity. For each material, the test is performed 3 times. The loss in mass during drying is measured after 15 minutes, 30 minutes and 60 minutes. The lost mass is expressed according to the following calculation:

$$\% wasted mass = \frac{{m}_{t 0} - {m}_{tx}}{{m}_{t 0}} \times 100 \qquad [ Math . 1 ]$$

With mₜₓ corresponding to the mass remaining at the measured time (t15 min, t30 min or t60 min) and mₜ₀ corresponding to the initially applied mass.

The volatile hydrocarbon oil is preferably chosen from linear or branched alkanes comprising from 8 to 19 carbon atoms and mixtures thereof.

As linear or branched C8-C19 alkanes, mention may especially be made of:
C8-C16 iso-alkanes (also known as isoparaffins) such as isododecane, isodecane, isohexadecane, hemi-squalane and, for example, oils sold under the tradenames ISOPAR® or PERMETYL®;
linear alkanes having, respectively,
C9-C17, C10-C14 hydrocarbon chains; such as a mixture of undecane and tridecane, marketed by BASF Care Creations under the name Cetiol® Ultimate;
   C15-C19 hydrocarbon chains, such as those marketed by Seppic under the name EMOGREEN® L15;
   C9-C12, C12-C14 hydrocarbon chains, such as those marketed by BIOSYNTHIS under the name VEGELIGHT® SILK (INCI name C9-12 ALKANE), VEGELIGHT® 1214LC;
   n-dodecane (C12) and n-tetradecane (C14), sold in particular by Sasol under the references PARAFOL® 12-97 and PARAFOL® 14-97, respectively;
   and mixtures thereof.

Thus, according to a particular embodiment, the branched C8-C19 alkanes are chosen from the group consisting of isododecane, isodecane, isohexadecane, hemi-squalane and mixtures thereof, preferably isododecane, hemi-squalane and the linear C8-C19 alkanes are chosen from the group consisting of n-dodecane, n-undecane, n-tridecane, n-tetradecane, n-pentadecane and mixtures thereof.

According to a particular embodiment, the composition comprises at least isododecane as branched C8-C19 alkane.

The total content of linear or branched alkanes makes it possible to dissolve the polyurethane silicone polymer and the phenyl silicone oil(s) of the composition, so that the homogeneous composition obtained permits applying a film to the lips which has particularly remarkable properties of lasting and gloss.

According to one particular embodiment, the content of linear or branched C8-C19 alkanes in the composition of the invention will be at least 20% by weight relative to the total weight of the composition. In particular, it will especially range from 20% to 60% by weight, in particular from 25% to 45% by weight and even more particularly from 30% to 40% by weight relative to the total weight of the composition.

### Phenyl Silicone Oil(s)

‘Silicone oil’ according to the invention is understood to mean an oil comprising at least one silicon atom, and especially at least one Si—O group.

‘Phenyl silicone oil’ according to the invention is understood to mean an organopolysiloxane substituted by at least one phenyl group.

As phenyl silicone oils having a refractive index of at least 1.45, in particular a refractive index at 25° C. greater than 1.45, preferably greater than or equal to 1.47, which can be used in the composition of the invention, mention may be made in particular of the oils of the name INCI phenylpropyldimethylsiloxysilicate, diphenyl dimethicone, diphenylsiloxyphenyltrimethicone, trimethyl pentaphenyl trisiloxane, phenyltrimethicone, trimethylsiloxyphenyl dimethicone and mixtures thereof.

The following references may be mentioned in particular:

TABLE 1

| | | Refractive index | Viscosity |
|---|---|---|---|
| tradename | INCI name | at 25° C.* | at 25° C.* (MPa · s) |
| Silshine 151 | Phenylpropyldimethylsiloxysilicate | 1.51-1.52 | 200-1000 |
| KF-54 HV | Diphenyl dimethicone | 1.501 | 4000-6000 |
| KF-56A | diphenylsiloxyphenyltrimethicone | 1.498 | 10-20 |
| Silshine 150 | Diphenyl dimethicone | 1.50 | 15-30 |
| DOW | Trimethyl pentaphenyl trisiloxane | 1.57-1.58 | 160-190 |
| CORNING | | | |
| PH-1555 | | | |
| SeraSense ® | Phenyltrimethicone | 1.46 | 15-30 |
| SF PTM | | | |
| 30 cst | | | |
| Belsil PDM- | Trimethylsiloxyphenyl | 1.46 | 925-1075 |
| 1000 | dimethicone | | |

*according to supplier sheets

According to one particular embodiment of the invention, the composition comprises at least one phenyl silicone oil with a refractive index greater than 1.50, or even greater than or equal to 1.55.

Thus, according to a particular embodiment, the composition of the invention comprises one or more phenyl silicone oil(s) with a refractive index ranging from 1.46 to 1.50 (e.g.: diphenyl dimethicone, diphenylsiloxyphenyltrimethicone, trimethylsiloxyphenyl dimethicone) and one or more phenyl silicone oil(s) with a refractive index greater than 1.50, in particular greater than 1.55 (e.g.: trimethylpentaphenyl trisiloxane).

According to a particular embodiment of the invention, the composition comprises at least one phenyl silicone oil having a viscosity at 25° C. of greater than 100 mPa·s. According to a particular and preferred embodiment, this phenyl silicone oil has a viscosity ranging, in particular, from 120 mPa·s to 1500 mPa·s, and preferably from 150 mPa·s to 1100 mPa·s. Such an oil can be called a glossy oil.

Mention may especially be made of BELSIL PDM 1000 from WACKER, phenyl silicone oils, such as those identified by the INCI name phenyl trimethicone, an example of which consists of the silicone available under the tradename MIRASIL PTM from RHODIA, those identified by the INCI name phenylpropyldimethylsiloxysilicate, an example of which consists of the silicone available under the tradename SILSHINE 151 from GENERAL ELECTRIC and those identified by the INCI name trimethyl pentaphenyl trisiloxane, an example of which consists of the silicone available under the tradename DC PH 1555 HRI from DOW CORNING, and mixtures thereof. Thus, the composition may preferably comprise a phenyl silicone oil of formula (VI)

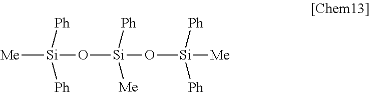

wherein Me represents methyl and Ph represents phenyl, also known as trimethyl pentaphenyl trisiloxane.

Such a phenyl silicone is manufactured in particular by Dow Corning under the reference PH-1555 HRI or Dow Corning 555 Cosmetic Fluid (chemical name: 1,3,5-trimethyl 1,1,3,5,5-pentaphenyl trisiloxane, INCI name: trimethyl pentaphenyl trisiloxane). The reference Dow Corning 554 Cosmetic Fluid can also be used. According to a preferred embodiment, the reference PH-1555 HRI (INCI name: trimethyl pentaphenyl trisiloxane) will be used.

According to another particular embodiment, the composition of the invention comprises at least one phenyl silicone oil of formula (VII)

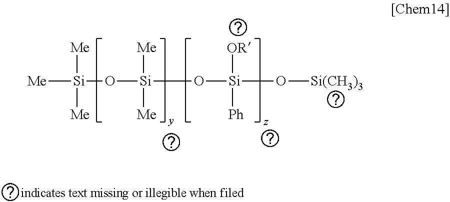

wherein
Me is methyl and Ph is phenyl, OR′ represents a —OSiMe₃ group;
y varies between 1 and 1000; and
z varies between 1 and 1000.

Belsils PDM20, PDM100 and PDM1000 from Wacker can be used, for example, and, in particular, trimethyl siloxyphenyl dimethicone, sold under the name BELSIL PDM 1000 marketed by Wacker.

According to still another particular embodiment, the phenyl silicone oil is an oil of formula (VIII)

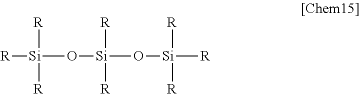

Wherein the R groups independently represent an —SiO(CH₃)₃ group, a methyl group or a phenyl group, the total number of phenyl being at least equal to 2, preferably equal to 3.

Mention may be made in particular of the oil sold under the names KF50, KF53, K54 and KF56 from Shin Etsu, preferably the reference KF56 of INCI name dipenylsiloxy phenyl trimethicone, the formula of which is the following:

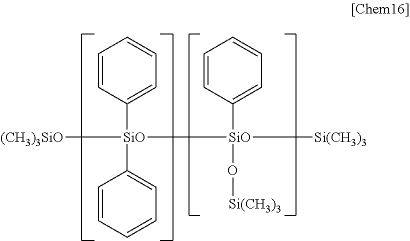

According to a particular and preferred embodiment, the composition of the invention will comprise two phenyl silicone oils.

Thus, according to a particular embodiment, the composition of the invention may comprise at least one trimethyl pentaphenyl trisiloxane, and, optionally, also a diphenylsiloxy phenyl trimethicone.

According to a particular embodiment, the total content of phenyl silicone oil(s) in the composition of the invention will especially range from 5 to 50% by weight, in particular from 8 to 30% by weight, relative to the total weight of the composition.

The person skilled in the art will be able to adjust the contents of phenyl silicone oil(s) in the ternary mixture described previously and consisting of linear or branched C8-C19 alkanes/phenyl silicone oils with a refractive index of at least 1.45/polyurethane silicone polymer, in order to obtain a translucent or transparent ternary mixture.

### Additional Compounds

According to a particular and preferred embodiment, the composition may also comprise a copolymer comprising carboxylate groups and polydimethylsiloxane groups, in order to further improve the gloss of the composition.

‘Copolymer comprising carboxylate groups and polydimethylsiloxane groups’ is understood to mean, according to the invention, a copolymer resulting from the polymerization (a) of one or more carboxylic monomer(s) (acid or ester), with (b) one or more polydimethylsiloxane (PDMS) chain(s) comprising at least one polymerizable radical.

As ‘carboxylic monomers’ suitable for the implementation of the invention, mention may be made of carboxylic acid monomers and carboxylic acid ester monomers.

According to one embodiment of the invention, the monomers in the form of esters may be chosen from linear or branched alkyl acrylates and/or methacrylates, preferably C1-C24 and better still C1-C22 alkyl, the alkyl radical being chosen from methyl, ethyl, stearyl, butyl and 2-ethylhexyl radicals, and mixtures thereof.

According to one embodiment of the invention, the copolymer may comprise, as carboxylate groups, at least one group chosen from acrylic acid, methacrylic acid, methyl, ethyl, stearyl, butyl and 2-ethylhexyl acrylates or methacrylates, and mixtures thereof.

‘Polydimethylsiloxanes’ (also called organopolysiloxanes or abbreviated PDMS) is understood to mean any organosilicon polymer or oligomer with a linear structure, of variable molecular weight, obtained by polymerization and/or polycondensation of suitably functionalized silanes, and consisting essentially of, by a repetition of main units in which the silicon atoms are linked together by oxygen atoms (═Si—O—Si═ siloxane bond), comprising trimethyl radicals directly linked by means of a carbon atom to said silicon atoms.

The copolymers which can be used in the composition of the invention can be obtained according to the usual methods of polymerization and grafting, for example by radical polymerization (a) of a PDMS comprising at least one polymerizable radical group (for example, on one of the ends of the chain or on both) and (b) at least one carboxylic monomer, as described, for example, in documents U.S. Pat. No. 5,061,481 and U.S. Pat. No. 5,219,560. The copolymers obtained may have a molecular weight ranging from approximately 3000 g/mol to approximately 200,000 g/mol and especially from approximately 5000 g/mol to approximately 100,000 g/mol.

A copolymer which can be used in a composition of the invention can be provided as such, or in dispersed form in a solvent, such as lower alcohols comprising from 2 to 8 carbon atoms, such as isopropyl alcohol, or oils such as volatile silicone oils (for example dimethicone).

As copolymers which can be used in a composition of the invention, mention may be made, for example, of acrylic acid and stearyl acrylate copolymers containing polydimethylsiloxane grafts, stearyl methacrylate copolymers containing polydimethylsiloxane grafts, acrylic acid and stearyl methacrylate copolymers containing polydimethylsiloxane grafts, or copolymers of methyl methacrylate, butyl methacrylate, 2-ethylhexyl acrylate and stearyl methacrylate with polydimethylsiloxane grafts. As copolymers which can be used in the composition of the invention, mention may also be made of the copolymers sold by SHIN-ETSU under the names KP-561 (CTFA name: acrylates/dimethicone), KP-541, where the copolymer is dispersed at 60% by weight in isopropyl alcohol (CTFA name: acrylates/dimethicone and isopropyl alcohol), acrylate/dimethicone copolymers in methyl trimethicone (such as, for example, KP-549 and KP-579 from Shin-Etsu), and acrylate/dimethicone copolymers in isododecane (such as, for example, KP-550 from Shin-Etsu).

According to a particular and preferred embodiment of the invention, an acrylate/dimethicone copolymer comprising a C18-C22 alkyl chain is used, such as acrylates/stearyl acrylate/dimethicone methacrylate (KP-561P from Shin Etsu) and acrylates/behenyl acrylate/dimethicone methacrylate (KP562P from Shin Etsu), preferably KP-561 P, which is in waxy form (melting point of approximately 40° C.).

The content of copolymer comprising carboxylate groups and polydimethylsiloxane groups in the composition of the invention will especially range from 0 to 20% by weight, in particular from 2 to 15% by weight, relative to the total weight of the composition.

According to a particular embodiment of the invention, the composition of the invention will comprise:
isododecane, preferably in a content ranging from 25% to 45% by weight relative to the total weight of the composition;
a trimethyl pentaphenyl trisiloxane and, optionally, also a diphenylsiloxy phenyl trimethicone, preferably in respective contents ranging from 5 to 15% by weight and from 5 to 15% by weight relative to the total weight of the composition, and a silicone-polyurethane polymer reaction product of a hydroxyl functionalized polyorganosiloxane of formula Ib:

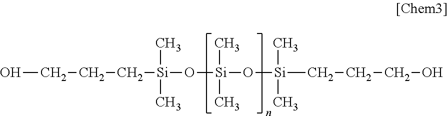

where n is as defined above with a methylene diphenyl diisocyanate, in particular in a content of 16 to 25% by weight of active material (dry matter) relative to the total weight of the composition;
and, advantageously, also a copolymer comprising carboxylate groups and polydimethylsiloxane groups.

### Other Additional Oils

The invention may also comprise other silicone or hydrocarbon oils, distinct from the oils described above, in order to provide lightness and lasting to the film deposited on the skin or comfort.

Mention may be made in particular of ester oils, such as esters of fatty acids, in particular of 4 to 22 carbon atoms, synthetic esters such as the oils of formula R₁COOR₂ wherein R₁ represents the residue of a linear or branched fatty acid containing from 4 to 40 carbon atoms and R₂ represents a hydrocarbon chain, especially branched, containing from 4 to 40 carbon atoms, provided that R₁+R₂ is ≤16, the hydroxylated esters, preferably having a total carbon number ranging from 35 to 70, the esters of fatty alcohol or of branched C24-C28 fatty acids, and mixtures thereof, preferably hydroxylated esters such as polyglyceryl-2 triisostearate, diisostearyl malate, and mixtures thereof.

The ester oils are particularly useful as dispersant for pigments, in particular pigments which comprise a surface treatment with a hydrophobic hydrocarbon compound aimed at conferring on these pigments an affinity for the fatty phase of the composition. The oil dispersing these treated pigments improves the homogeneity of the composition according to the invention and that of the film applied to the lips. The visual appearance is thus greatly improved.

The fatty phase of the composition of the invention is generally textured or structured with a gelling and/or texturing agent, in particular a lipophilic gelling agent, a wax, a pasty fatty substance, or mixtures thereof.

According to a particular embodiment, the composition of the invention comprises at least one lipophilic gelling agent.

### Gelling Agents

As lipophilic gelling agent, mention may especially be made of mineral gelling agents such as bentones dispersed in an oil. Mention may be made, for example, of reference BENTONE GEL ISD V from Elementis of INCI name Isododecane (and) Disteardimonium Hectorite (and) Propylene Carbonate.

Thus, according to a particular embodiment, the composition of the invention also comprises a lipophilic gelling agent, in particular a mineral gelling agent, preferably a bentone.

Other lipophilic gelling agents may be used in the composition of the invention if they still permit obtaining a solution as defined previously.

The total content of lipophilic gelling agent(s) may range from 0.1 to 5%, in particular from 0.5 to 3% by weight relative to the total weight of the composition.

Thus, according to a particular embodiment of the invention, the composition of the invention will comprise:
isododecane, preferably in a content ranging from 25% to 45% by weight relative to the total weight of the composition;
a trimethyl pentaphenyl trisiloxane and, optionally, also a diphenylsiloxy phenyl trimethicone, preferably in respective contents ranging from 5 to 15% by weight and from 5 to 15% by weight relative to the total weight of the composition; and
a silicone-polyurethane polymer reaction product of a polyorganosiloxane functionalized by hydroxyl groups of formula Ib:

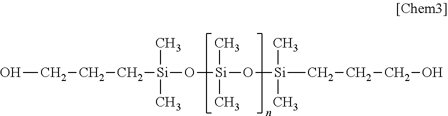

where n is as defined above with a methylene diphenyl diisocyanate, in particular in a content of 16 to 25% by weight of active material (dry matter) relative to the total weight of the composition;
a bentone-type lipophilic gelling agent (INCI name Disteardimonium Hectorite), preferably in a content ranging from 0.1 to 5%, relative to the total weight of the composition;
and, advantageously, also a copolymer comprising carboxylate groups and polydimethylsiloxane groups.

The composition according to the invention may also comprise an ingredient chosen from a wax, a filler, a colouring agent and mixtures thereof.

### Waxes

Within the meaning of the present invention, ‘wax’ is intended to designate a compound solid at 25° C. which exhibits a reversible solid/liquid state change and a melting temperature greater than 30° C., preferably greater than 45° C.

Mention may be made of natural waxes (sunflower wax, berry wax), microcrystalline waxes, paraffin waxes, polyethylene waxes, ozokerite, carnauba wax, beeswax, and mixtures thereof.

Thus, according to a particular and preferred embodiment, the composition of the invention will also comprise at least one wax, making it possible to stabilize, or even to freeze, the system without disturbing the lasting and gloss result.

A composition according to the invention may comprise a wax content ranging from 0.5% to 10%, for example from 1% to 5% by weight, relative to the total weight of the composition.

According to a particular embodiment, the composition of the invention also comprises an additional ingredient chosen from fillers, colouring agents and mixtures thereof.

### Fillers

In addition, the composition of the invention may advantageously comprise fillers to provide comfort and prevent the possible appearance of stickiness linked to the high level of polymer held in the composition.

Within the meaning of the invention, ‘fillers’ should be understood to mean colourless or white particles of any shape, of mineral or organic, natural or synthetic nature, which are in a form (plate, spherical or oblong) which is insoluble and dispersed in the medium of the composition. The fillers are chosen in particular from silicas, micas, of natural or synthetic origin, kaolin, zinc and titanium oxides; calcium carbonate, magnesium carbonate and hydrogen carbonate; zinc, magnesium or lithium stearate, zinc laurate, magnesium myristate; powders of synthetic polymers, such as polyethylene, polyesters, polyamides (e.g. nylon); polyacrylic or polymethacrylic acid powders, silicone or silicone resin powders; cellulose powders; mineral powders such as spherical silica; spherical titanium dioxides; glass and ceramic beads; powders of organic materials of natural origin such as corn, wheat, rice and cotton starches, crosslinked or otherwise, and mixtures thereof.

Preferred fillers especially include silica powders, cellulose powders, cotton powders and mixtures thereof.

The fillers may be present in a content ranging from 2% to 10%, in particular from 3% to 8% by weight relative to the total weight of the composition.

A composition of the invention is in particular a make-up composition comprising at least one colouring agent.

### Colouring Agents

Withing the meaning of the present invention, ‘colouring agent’ is understood to mean a compound capable of producing a coloured optical effect when it is formulated in a sufficient amount in an appropriate cosmetic medium.

A colouring agent may be chosen from organic or inorganic colouring agents, materials with an optical effect, and mixtures thereof, which are soluble or insoluble in the continuous phase of the invention, in this case the oil phase of the invention.

According to a particular embodiment, the colouring agent or agents are especially chosen from mineral pigments, organic pigments and mixtures thereof.

‘Pigments’ is understood to mean white or coloured, mineral or organic particles, insoluble in an aqueous solution, intended to colour and/or opacify the resulting deposit. Mention may be made of mineral pigments, organic pigments and composite pigments (i.e., pigments based on mineral and/or organic materials).

As ‘mineral pigments’, mention may be made, by way of examples, of titanium dioxide (rutile or anatase), optionally surface-treated; black, yellow, red and brown iron oxides; manganese violet; blue ultramarine chromium oxide, hydrated chromium oxide and ferric blue.

For compositions intended for the lips, mention may be made, by way of examples, of titanium dioxide; black, yellow, red and brown iron oxides and manganese violet.

As ‘organic pigments’, mention may be made, for example, of the pigments D Et C Red No. 19; D Et C Red No. 9; D Et C Red No. 22; D Et C Red No. 21; D Et C Red No. 28; D Et C Yellow No. 6; D Et C Orange No. 4; D Et C Orange No. 5; D Et C Red No. 27; D Et C Red No. 13; D Et C Red No. 7; D Et C Red No. 6; D Et C Yellow No. 5; D Et C Red No. 36; D Et C Red No. 33; D Et C Orange No. 10; D Et C Yellow No. 6; D Et C Red No. 30; D EtC Red No. 3; D EtC Blue 1; carbon black and lacquers.

In particular, the at least one colouring agent is present in the composition in a content ranging from 2% to 20% by weight, preferably from 3% to 15% by weight, relative to the total weight of the composition.

### FORMULATIONS

The composition of the invention is a make-up and/or care composition for keratin materials, in particular a make-up and/or care composition for the skin and/or the lips, preferably the lips.

According to a first embodiment, the composition of the invention is a top coat composition, which can be used over any make-up composition for the lips, in order to provide lasting and shine.

According to another embodiment, the composition of the invention is single-step make-up composition, to provide colour, shine and lasting.

The composition of the invention is in particular a liquid composition, preferably for the lips.

In one embodiment of the invention, it may be in liquid form and may be a fluid lipstick, a lip lacquer, a lip gloss, a sunscreen or skin colouring product, an eyeshadow or even an eyeliner.

Preferably, the composition is a make-up composition for the lips, in particular in the form of a gloss.

### COSMETIC PROCESS

The invention also relates to a cosmetic process for caring for and/or applying make-up to keratin materials, comprising the application to said keratin materials, in particular the skin and/or the lips, preferably the lips, of a composition according to the invention.

The composition applied to the lips is preferably a liquid composition for applying make-up to the lips, in particular a fluid lipstick, a lip lacquer or a gloss.

The process according to the invention is, in particular, a process for applying make-up to the lips, intended to deposit on said lips, in a single step, a glossy film with a long-lasting gloss and colour.

The invention will now be illustrated in the following non-limiting examples. The % are expressed as % by weight relative to the total weight of the composition, unless otherwise indicated.

### EXAMPLES

### Example 1: Solubility Tests

Solubility tests are performed for different mixtures comprising silicone polyurethane polymer, a volatile hydrocarbon oil and at least one phenyl silicone oil. The ratios for which a clear and transparent solution is obtained are those which are suitable for the invention. In this case, the compounds of the mixture form only a single phase. In the case where the ratios of the compounds of the mixture do not make it possible to obtain a clear oil phase, an opacity of the mixture is observed, meaning that the compounds are immiscible with the ratios tested.
For these tests, SILMER UR1550 is used, which comprises silicone polyurethane polymer (INCI=BIS-Hydroxypropyl Dimethicone/SMDI Copolymer) dispersed in isododecane, containing 45% by dry weight of polymer.
Depending on the tested ratio, an additional quantity of the ingredient is added.
1. Mixtures (with 1 phenyl silicone oil with viscosity >150 mPa·s at 25° C.): BIS-Hydroxypropyl Dimethicone/SMDI Copolymer/Isododecane/Trimethyl Pentaphenyl Trisiloxane (DOW CORNING PH-1555)

The mixtures below comprise 40% by weight of SILMER UR-1550, i.e., 18% by dry weight of silicone polyurethane polymer and 22% by weight of isododecane. The 18% of silicone polyurethane polymer is dispersed in 82% of a mixture of oils comprising an isododecane and a phenyl silicone oil with a viscosity of 160 mPa·s at 25° C.

The results are included in Table 2 below:

TABLE 2

| Compound | INCI name | % | | | |
|---|---|---|---|---|---|
| Silicone polyurethane | BIS-Hydroxypropyl | 18 | | | |
| polymer | Dimethicone/SMDI | | | | |
| | Copolymer | | | | |
| Linear or branched C8-C19 | Isododecane | 42 | 52 | 62 | 72 |
| alkane | | (22 + 20) | (22 + 30) | (22 + 40) | (22 + 50) |
| | | or less | | | |
| Phenyl silicone oil with | Trimethyl Pentaphenyl | 40 or | 30 | 20 | 10 |
| refractive index >1.45 and | Trisiloxane (DOW | more | | | |
| viscosity 160-190 mPa · s at | CORNING PH-1555)) | | | | |
| 25° C. | | | | | |
| | TOTAL | 100 | 100 | 100 | 100 |
| Appearance of the | | opaque | clear | clear | clear |
| mixture | | | | | |

Observation of the Appearance of the Mixture:
Opaque=immiscible
Clear=miscible=OK for the invention

The results show that, in order to obtain a clear mixture with 18% by weight of silicone polyurethane polymer, it is necessary, in the case of a phenyl silicone oil with a viscosity >100 mPa·S at 25° C., to have a mixture of oils comprising 5 to 35% of phenyl silicone oil with a viscosity of >150 mPa·s and 45 to 75% of C8-C19 alkanes, based on the total weight of the mixture. In this case, the content of C8-C19 alkanes will be at least 1.5 times greater than the said content of phenyl silicone oil with a viscosity >150 mPa·S at 25° C.

Reference will be made more generally (rounding 18% to 20% silicone polyurethane polymer) to a mixture of 80 (oils)/20 silicone polymer polyurethane, with a weight ratio of 5-35% phenyl silicone oil(s) with a viscosity of >100 mPa·s/45-75% C8-C19 alkanes/20% silicone polymer polyurethane, or a weight ratio of 1/3 phenyl silicone oils(s) with a viscosity of >100 mPa s and 2/3 C8-C19 alkanes relative to the total weight of the oils in the mixture.

Mixtures (with 2 Phenyl Silicone Oils):
Bis-Hydroxypropyl Dimethicone/SMDI Copolymer/Isododecane/Trimethyl Pentaphenyl Trisiloxane (DOW CORNING PH-1555)/Diphenylsiloxyphenyltrimethicone (KF-56A)

The results are included in Table 3 below

TABLE 3

| Compound | INCI name | % | | |
|---|---|---|---|---|
| Silicone polyurethane | BIS-Hydroxypropyl | 18 | | |
| polymer | Dimethicone/SMDI | | | |
| | Copolymer | | | |
| Linear or branched C8-C19 | Isododecane | 22 | | |
| alkane | | | | |
| Phenyl silicone oil with | Trimethyl Pentaphenyl | 30 or | 20 | 10 |
| refractive index >1.45 and | | more | | |
| viscosity 160-190 mPa · s at | Trisiloxane (DOW CORNING | | | |
| 250° C. | PH-1555)) | | | |
| Phenyl silicone oil with | Diphenylsiloxyphenyltrimethicone | 30 or | 40 | 50 |
| refractive index >1.45 and | (KF-56A) | less | | |
| viscosity 10-20 mPa · s at | | | | |
| 25° C. | | | | |
| | TOTAL | 100 | 100 | 100 |
| Appearance of the mixture | | opaque | clear | clear |

Observation of the Appearance of the Mixture:
Opaque=immiscible
Clear=miscible=OK for the invention

The results show that, in order to obtain a clear mixture with 18% by weight of silicone polyurethane polymer, it is necessary, in the case of a combination of phenyl silicone oil with a viscosity >100 mPa·s and phenyl silicone oil with a viscosity <50 mPa·s, to have a mixture of oils comprising 60% of phenyl silicone oil(s) and 20% of C8-C19 alkanes, based on the total weight of the mixture. In such a mixture, the content of low-viscosity phenyl silicone oil is greater than the content of high-viscosity phenyl silicone oil and the content of C8-C19 alkanes remains greater than or equal to the content of high-viscosity phenyl silicone oil.

Reference will be made more generally (rounding 18% to 20% silicone polyurethane polymer) to a 80 (oils)/20 silicone polyurethane polymer mixture, with a weight ratio of 60% phenyl silicone oil(s)/20% C8-C19 alkanes/20% silicone polyurethane polymer. The combination of a phenyl silicone oil of low viscosity with a phenyl silicone oil of high viscosity thus makes it possible to incorporate into the oily mixture a greater total content of phenyl silicone oils than in the preceding example, for a still more improved gloss result.

These results show that the low solubility of the polyurethane silicone polymer in the presence of phenyl silicone oil requires the addition of an excess of hydrocarbon oil (isododecane in the present examples) in order to obtain a clear solution.

Among the phenyl silicone oils tested, the presence of KF-56A in the mixture makes it possible to increase the total content of phenyl silicone oils and therefore the result of gloss of the film.

### Example 2: Liquid Lipstick

The composition below is a liquid lipstick according to the invention.

The percentages are expressed by weight relative to the final composition.

TABLE 4

| | Commercial | | |
|---|---|---|---|
| | references | Ingredients | % |
| A | SILMER UR-5050 | ISODODECANE 22.0 | 40 |
| | | BIS-HYDROXYPROPYL DIMETHICONE/SMDI | |
| | | COPOLYMER 18.0 | |
| A | BENTONE GEL ISD | ISODODECANE 13.05 | 15 |
| | V MB | DISTEARDIMONIUM HECTORITE 1.5 | |
| | | PROPYLENE CARBONATE 0.45 | |
| A | KP-561 P | ACRYLATES/STEARYL | 6 |
| | | ACRYLATE/DIMETHICONE METHACRYLATE | |
| | | COPOLYMER | |
| A | KF-56A | DIPHENYLSILOXY PHENYL TRIMETHICONE | 6 |
| A | DOW CORNING PH- | TRIMETHYL PENTAPHENYL TRISILOXANE | 6 |
| | 1555 HRI | | |
| B | SALACOS 222 | DIISOSTEARYL MALATE | 15.4 |
| B | KAHLWAX 6607L MB | HELIANTHUS ANNUUS (SUNFLOWER) SEED | 2 |
| | | WAX 1.967 | |
| | | ASCORBYL PALMITATE 0.011 | |
| | | TOCOPHEROL 0.011 | |
| | | HELIANTHUS ANNUUS (SUNFLOWER) SEED | |
| | | OIL 0.011 | |
| B | | Preservatives | 0.2 |
| C | | PIGMENTS | 4.4 |
| D | AMILON | SILICA 4.75 (and) LAUROYL LYSINE 0.25 | 5 |
| | | TOTAL | 100 |

The composition is Prepared According to the Following Procedure:

The SILMER, KP 561P and oils of phase A are mixed under Rayneri at 70° C. until a clear mixture is obtained. The BENTO GEL is then added and the mixture is left to homogenize at 1500 RPMs for the necessary time.

The pigments of phase C are dispersed in the ester oils of phase B, and ground. They are added together with the wax to the preceding phase. After cooling to below 40° C., the silica filler is added to the preceding phase and the mixture is left to homogenize for 5 minutes.

The composition is a liquid and homogeneous lip product.

When applied to the lips, the composition makes it possible to obtain a long-lasting and glossy make-up in a single step.

### Example 3: Lipstick

The composition below is a liquid lipstick according to the invention.

The percentages are expressed by weight relative to the final composition.

TABLE 5

| | Commercial | | |
|---|---|---|---|
| | references | Ingredients | % |
| A | SILMER UR-5050 | ISODODECANE 22.0 | 40 |
| | | BIS-HYDROXYPROPYL DIMETHICONE/SMDI | |
| | | COPOLYMER 18.0 | |
| A | BENTONE GEL ISD | ISODODECANE 13.05 | 15 |
| | V MB | DISTEARDIMONIUM HECTORITE 1.5 | |
| | | PROPYLENE CARBONATE 0.45 | |
| A | KF-56A | DIPHENYLSILOXY PHENYL TRIMETHICONE | 5 |
| A | SILSHINE ® 151 | PHENYLPROPYLDIMETHYLSILOXYSILICATE | 10 |
| B | SALACOS 222 | DIISOSTEARYL MALATE | qs 100 |
| B | | Preservatives | 0.2 |
| C | UNIPURE; | Pigments | 5.2 |
| | SUNCROMA FDC | | |
| | | TOTAL | 100 |

The composition is prepared according to the following procedure:

The SILMER, KP 561P and oils of phase A are mixed under Rayneri at 70° C., until a clear mixture is obtained. The BENTO GEL is then added and the mixture is left to homogenize at 1500 RPMs for the necessary time.

The pigments of phase C are dispersed in the ester oils of phase B and ground. They are added to phase A. The composition is then cooled to room temperature.

The composition is a liquid and homogeneous lip product.

When applied to the lips, the composition makes it possible to obtain a long-lasting and glossy make-up in a single step.

### Example 4: Lipstick

The composition below is a liquid lipstick according to the invention.

The percentages are expressed by weight relative to the final composition.

TABLE 6

| | | Ingredient | % |
|---|---|---|---|
| A | SILMER UR-5050 | ISODODECANE 22.0 | 40 |
| | | BIS-HYDROXYPROPYL DIMETHICONE/SMDI | |
| | | COPOLYMER 18.0 | |
| A | BENTONE GEL ISD | ISODODECANE 13.05 | 15 |
| | V MB | DISTEARDIMONIUM HECTORITE 1.5 | |
| | | PROPYLENE CARBONATE 0.45 | |
| A | KP-561 P | ACRYLATES/STEARYL ACRYLATE/DIMETHICONE | 10 |
| | | METHACRYLATE COPOLYMER | |
| A | KF-56A | DIPHENYLSILOXY PHENYL TRIMETHICONE | 10 |
| B | SALACOS 222 | DIISOSTEARYL MALATE | qs 100 |
| B | | Preservatives | 0.2 |
| C | UNIPURE; | Pigments | 02.5 |
| | SUNCROMA FDC | | |
| | | TOTAL | 100 |

The composition is prepared according to the following procedure:

The SILMER, KP 561P and oils of phase A are mixed under Rayneri at 70° C., until a clear mixture is obtained. The BENTO GEL is then added and the mixture is left to homogenize at 1500 RPMs for the necessary time.

The pigments of phase C are dispersed in the ester oils of phase B, and ground. They are added to phase A. The composition is then cooled to room temperature.

The composition is a liquid and homogeneous lip product.

When applied to the lips, the composition makes it possible to obtain a long-lasting and glossy make-up in a single step.

### Example 5: Top Coat

A non-coloured composition according to the invention is prepared. The formula is shown in the table below.

TABLE 7

| | Invention |
|---|---|
| | example |
| Ingredient (INCI name) | % by weight |
| SILMER ® UR-1550 | 40 |
| Bis- | |
| hydroxypropyl dimethicone/SMDI copolymer | |
| Isododecane | |
| BELSIL ® PDM 1000 | 6 |
| Trimethylsiloxyphenyl dimethicone | |
| AMILON ® | 5 |
| Silica (and) Lauroyl lysine (95/5) | |
| BENTONE ® GEL | 25 |
| Disteardimonium hectorite | |
| Propylene carbonate | |
| Isododecane | |
| Isododecane | 24 |

SILMER® UR-1550 comprises Bis-hydroxypropyl dimethicone/SMDI copolymer containing 45% by weight of dry matter in isododecane.

AMILON: Silica (and) Lauroyl lysine (95/5)

The composition is prepared according to the following protocol:

The composition is applied to the lips to form a film. It may advantageously be used as a top coat to provide shine and shine lasting after the application of a first layer of a coloured film on the lips.

1. A homogeneous cosmetic composition for applying make-up to keratin materials, comprising, in a physiologically acceptable medium, at least:
one or more linear or branched C8-C19 alkane(s);
one or more phenyl silicone oil(s) whose refractive index at 25° C. is greater than 1.45, ; and
a silicone-polyurethane polymer.

2. The composition according to claim 1, wherein the composition is a liquid and anhydrous composition.

3. The composition according to claim 1 wherein the silicone-polyurethane polymer is present in the composition in an active material content ranging from 14 to 25%, by weight relative to the total weight of the said composition.

4. The composition according to claim 1, further comprising a lipophilic gelling agent.

5. The composition according to claim 1, wherein the branched C8-C19 alkanes are chosen from the group consisting of isododecane, isodecane, isohexadecane, hemi-squalane and mixtures thereof, and the linear C8-C19 alkanes are chosen from the group consisting of n-dodecane, n-undecane, n-tridecane, n-tetradecane, n-pentadecane and mixtures thereof.

6. The composition according to claim 1, wherein the silicone oil(s) comprising at least one phenyl group, whose refractive index at 25° C. is greater than 1.45, are chosen from the group consisting of oils of the INCI names phenylpropyldimethylsiloxysilicate, diphenyldimethicane, diphenylsiloxyphenyltrimethicone, trimethyl pentaphenyl trisiloxane, phenyltrimethicone, trimethylsiloxyphenyl dimethicone, and mixtures thereof.

7. The composition according to claim 1, wherein the composition comprises at least one trimethyl pentaphenyl trisiloxane.

8. The composition according to claim 1, wherein the silicone-polyurethane polymer is the reaction product of a polyorganopolysiloxane functionalized by hydroxyl groups, corresponding to the structure of formula (I) 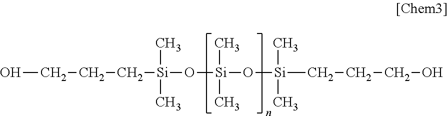
wherein n is a whole number between 0 and 5000;
with a diisocyanate compound.

9. The composition according to claim 1, further comprising a copolymer comprising carboxylate groups and polydimethylsiloxane groups.

10. The composition according to claim 1, wherein the composition comprises:
isododecane;
a trimethyl pentaphenyl trisiloxane, and
a silicone-polyurethane polymer reaction product of a hydroxyl functionalized polyorganosiloxane of formula Ib: 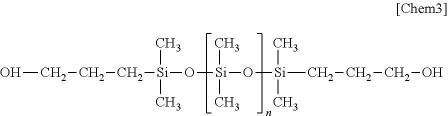
where n is as defined above with a methylene diphenyl diisocyanate.

11. The composition according to claim 1, further comprising an ingredient selected from a wax, a filler, a colouring agent and mixtures thereof.

12. The composition according to claim 11, wherein the fillers are chosen from the group consisting of silica powders, cellulose powders, cotton powders and mixtures thereof.

13. A cosmetic process for caring for and/or applying make-up to keratin materials, comprising the application, to the said keratin materials, of a composition according to claim 1.

14. The composition according to claim 1, further comprising at least one pigment.

15. The composition according to claim 8, further comprising a diphenylsiloxy phenyl trimethicone.
